(19) United States
(12) Patent Application Publication
(10) Pub. No.: US 2022/0062152 A1
(43) Pub. Date: Mar. 3, 2022

(54) COSMETIC CLEANSING COMPOSITION COMPRISING A COPOLYMER OF THE ASE OR HASE TYPE

(71) Applicant: COATEX, Genay (FR)

(72) Inventor: Clementine CHAMPAGNE, Caluire-Et-Cuire (FR)

(73) Assignee: COATEX, Genay (FR)

(21) Appl. No.: 17/420,253

(22) PCT Filed: Jan. 23, 2020
(86) PCT No.: PCT/FR2020/000014

§ 371 Date: Jul. 1, 2021

(30) Foreign Application Priority Data
Jan. 23, 2019 (FR) 1900571

(51) Int. Cl. A61K 8/81; A61Q 19/10; A61K 8/34; A61K 8/19; A61Q 1/14

(52) U.S. Cl. CPC A61K 8/8152; A61Q 19/10; A61K 8/345; A61K 2800/30; A61K 8/19; A61Q 1/14; A61K 2800/33; A61K 8/347

(57) ABSTRACT

The invention relates to the field of cosmetic cleansing compositions and proposes an aqueous cosmetic cleansing composition which does not comprise any surfactant compound and no lipophilic compound. This composition comprises a water-soluble humectant compound and a copolymer of the ASE type or a copolymer of the HASE type. The invention also provides a cleansing method using this composition, and in particular a make-up removal method.

The invention relates to the field of cosmetic cleansing compositions and proposes a water-based cosmetic cleansing composition that does not comprise any surfactant compound or any lipophilic compound. This composition comprises a water-soluble moistening compound and an ASE copolymer or an HASE copolymer. The invention also provides a cleansing method using this composition, and in particular a makeup removal method.

In the field of cosmetic cleansing compositions, and particularly makeup removal compositions, there are generally three groups of technologies.

Water-based micellar solutions whose cleansing ability relies on the use of surfactant compounds. However, these water-based micellar solutions are not effective enough for makeup removal.

There are also cosmetic cleansing compositions in the form of milks that are emulsions in which droplets of a lipophilic compound are dispersed in a water-soluble substance, generally water. These cleansing milks can improve cleansing, particularly makeup removal, but they often lead to the formation of a greasy film on the skin. Removing such a film then requires the skin to be rinsed. Otherwise, an unpleasant sensation persists due to this film.

Lastly, there are biphasic formulations that attempt to combine these two technologies in order to avoid the drawbacks but which are unable to achieve this to a satisfactory extent and require the formulations to be shaken before use.

Moreover, if the cosmetic cleansing compositions contain surfactant compounds, their application on certain parts of the body can lead to irritation problems, which can lead to discomfort or even health risks.

In the field of cosmetic cleansing compositions, it is also important to supplement the cleansing activity with a moisturising or softening effect, especially when applied to the skin.

Cosmetic cleansing compositions must also be able to be combined with other ingredients, in particular cosmetic formulations, without losing their efficacy.

They must not be incompatible with the other ingredients in these formulations, in particular shower gel formulations or shampoo formulations.

When used, cosmetic cleansing compositions must be effective for cleansing different substrates. In particular, they must be effective in cleansing keratin substrates, particularly hair, body hair and skin.

The cosmetic cleansing compositions must also be pleasant to the touch or when applied; they must in particular leave a velvety, non-sticky and non-greasy sensation on the skin after use. They must also have a texture that allows them to be used easily and without any wastage. In particular, these cosmetic cleansing compositions must have a viscosity that is well-controlled. They must also have an attractive appearance, in particular with regard to their colour or their transparency.

Some of these cosmetic cleansing compositions must also be able to have good suspensive properties, especially if they contain particulates.

These cosmetic cleansing compositions must also be easy to prepare, in particular using a reduced number of ingredients.

Document EP 1 467 698 A1 describes hair setting compositions comprising associative polymers as a hair setting and rheology-modifying component. Document JP H08217627 relates to the stabilisation of a cosmetic emulsion by means of a carboxyvinyl polymer modified by an alkyl group. Document EP 0 705 595 A2 describes a water-based hair setting composition comprising an acrylic resin, water and iodopropynyl butyl carbamate as an antimicrobial agent. Document EP 3 346 984 A1 describes a liquid composition for personal hygiene comprising at least one surfactant alkyl ether sulphate, silicones and a structuring agent.

These compositions are not cleansing compositions.

The cosmetic cleansing compositions in the prior art do not provide satisfactory solutions to the various problems encountered. There is thus a need to have improved cosmetic cleansing compositions.

The invention thus provides a cosmetic cleansing composition that offers a solution to all or part of the problems of the cosmetic cleansing compositions in the prior art.

The water-based cosmetic cleansing composition according to the invention, which does not comprise any surfactant compound or any lipophilic compound, comprises:
(a) at least one water-soluble compound chosen among a water-soluble moistening compound, a water-soluble solvent and combinations thereof;
(b) at least one copolymer chosen among:
   (b1) an ASE copolymer,
   (b2) an HASE copolymer.

The water-based cosmetic cleansing composition according to the invention therefore does not comprise any surfactant compound or any lipophilic compound.

Preferably according to the invention, the composition according to the invention consists of water and:

(a) at least one water-soluble compound;

(b) at least one copolymer chosen among:
(b1) an ASE copolymer,
(b2) an HASE copolymer; optionally

(c) at least one preservative compound.

The compound (a) according to the invention is a water-soluble compound. It is generally a compound that can be mixed with water, at room temperature and in any proportion, without producing any dephasing. Preferably, the compound (a) according to the invention is a hydrophilic compound.

According to the invention, the compound (a) according to the invention is chosen among a water-soluble moistening compound, a water-soluble solvent and combinations thereof. In addition to being water-soluble, the compound (a) is chosen among substances capable of binding water molecules. These are usually hygroscopic substances. Advantageously, a moistening compound (a) makes it possible to maintain the water content of the composition according to the invention during its preparation or storage as well as during its application, in particular on the skin.

According to the invention, the compound (a) can be an emollient compound and can thus make it possible to soften or soothe inflamed tissues.

Preferably, the compound (a) according to the invention is different from ethanol. Also preferably, the compound (a) according to the invention is chosen among a synthetically-derived water-soluble compound, a naturally-derived water-soluble compound and combinations thereof. In particular, the compound (a) is a plant-based water-soluble compound. Advantageously, the water-soluble compound (a) is chosen among diols; triols; sugars; modified sugars; ethers; protein compounds; amino acids; triglycerides and combinations thereof; particularly a water-soluble compound (a) chosen among pidolic acid (PCA; CAS No. 98-79-3 L-form or S(−)-form; 4042-36-8 D-form or R(+)-form, 149-87-1 racemic form); PCA derivatives, particularly arginine PCA, chitosan PCA, copper PCA derivative, ethyl hexyl PCA, lauryl PCA, magnesium PCA derivative, sodium PCA derivative, zinc PCA derivative; butylene glycol; pentylene glycol; calcium gluconate; fructose; glucose; isomalt; lactose; maltitol; mannitol; polydextrose; sorbitol; saccharose; sucrose; xylitol; glycerol; glycerine (trihydroxypropane propane-1,2,3-triol or 1,2,3-propanetriol); glycyrrhizic acid; glycyrrhizic acid derivatives; histidine; hyaluronic acid; hyaluronic acid salts, particularly sodium hyaluronate, silk hydrolysate, keratin hydrolysate; soy hydrolysate, PEG-7; PEG-8; PEG-10; PEG-12; PEG-14; phytantriol; propylene glycol; silk (serica); urea; methylpropanediol; hexanetriol; capryl glycol; dipropylene glycol; erythritol; triethylene glycol; hexylene glycol; phytantriol hexanediol; beeswax triol; organic moisteners; panthenol; provitamin B5; inositol glycogen; sugars and modified sugars; polyglyceryl; sorbitol; honey; polymer polyols; inositol; vitamin B7, high-intensity-sweetening liquorice saponin; ethers; isoceteth-x; isolaureth-x; laneth-x; laureth-x; steareth-x; polyethylene glycols; polyethylene glycol derivatives; trideceth-(5-50); tridecanol polyethylene glycol ether; silicone copolyols; protein-based moistening compounds; cocodimonium hydroxypropyl hydrolysed casein; cocodimonium hydroxypropyl hydrolysed collagen; cocodimonium hydroxypropyl hydrolysed keratin; cocodimonium hydroxypropyl hydrolysed rice proteins; cocodimonium hydroxypropyl hydrolysed silk proteins; cocodimonium hydroxypropyl hydrolysed soy proteins; cocodimonium hydroxypropyl hydrolysed wheat proteins; cocodimonium hydroxypropyl silk amino acids; cocoyl hydrolysed collagen; cocoyl hydrolysed keratin; keratin; hydrolysed keratin; hydrolysed oat proteins; hydrolysed quinoa proteins; potassium cocoyl hydrolysed collagen; triethanolamine-cocoyl hydrolysed collagen; triethanolamine-cocoyl hydrolysed soy proteins; histidine; amino acids; triglycerides; glyceryl triacetate; glyceryl triacetate obtained by natural glycerine esterification; alpha-hydroxy acids; fruit sugar derivatives; milk sugar derivatives; fruit acids; lactic acid; neoagarobiose; Aloe vera.

More preferably, the water-soluble compound (a) is chosen among diols, triols, butylene glycol, pentylene glycol; glycerol; glycerine; propylene glycol; methylpropanediol; hexanetriol; capryl glycol; dipropylene glycol; erythritol; triethylene glycol; hexylene glycol; panthenol; protein-based moistening compounds and combinations thereof.

The composition according to the invention also comprises at least one copolymer (b) chosen among an ASE copolymer, an HASE copolymer and combinations thereof. According to the invention, “ASE” (alkali-swellable emulsion) refers to an alkali-soluble emulsion and “HASE” (hydrophobically-modified alkali-swellable emulsion) refers to a hydrophobically-modified alkali-soluble emulsion.

Preferably, the copolymer (b) is an ASE copolymer (b1) prepared by at least one polymerisation reaction

A—of at least one anionic monomer (A) comprising at least one polymerisable olefinic unsaturation, preferably an anionic monomer comprising at least one polymerisable olefinic unsaturation and at least one carboxylic acid group, preferably an anionic monomer chosen among acrylic acid, methacrylic acid, an acrylic acid salt, a methacrylic acid salt, and combinations thereof, optionally combined with maleic acid, itaconic acid, crotonic acid, a maleic acid salt, an itaconic acid salt, a crotonic acid salt and combinations thereof, much more preferentially acrylic acid, methacrylic acid and combinations thereof; and

B—of at least one ester (B) of an acid chosen among acrylic acid, methacrylic acid, maleic acid, itaconic acid and crotonic acid, preferably an acrylic acid ester or a methacrylic acid ester, more preferentially a C₁-C₇ ester; even more preferentially an ester chosen among methyl acrylate, ethyl acrylate, propyl acrylate, butyl acrylate, ethyl hexyl acrylate, methyl methacrylate, ethyl methacrylate, propyl methacrylate, butyl methacrylate, ethyl hexyl methacrylate, and combinations thereof.

More preferably according to the invention, the anionic monomer (A) is chosen among acrylic acid, methacrylic acid, an acrylic acid salt, a methacrylic acid salt, and combinations thereof.

Also preferably according to the invention, the compound (B) is chosen among methyl acrylate, ethyl acrylate, butyl acrylate, ethyl hexyl acrylate, methyl methacrylate, ethyl methacrylate and combinations thereof.

Particularly preferably, the copolymer (b) is an HASE copolymer (b2) prepared by polymerisation reaction
A—of at least one anionic monomer (A);
B—at least one ester (B) and
C—of at least one compound (C) of formula (I):

R¹-(EO)ₘ—(PO)ₙ—R² (I)

wherein:
m and n, identical or different, independently represent 0 or an integer or decimal less than 150, m or n being different from 0,
EO independently represents a CH₂CH₂O group,
PO independently represents a group chosen among CH(CH₃)CH₂O and CH₂CH(CH₃)O and combinations thereof,
R¹ independently represents a group comprising at least one polymerisable olefinic unsaturation, preferably a group chosen among an acrylate group, a methacrylate group, an unsaturated urethane group, in particular acrylurethane, methacrylurethane, α-α′-dimethyl-isopropenyl-benzylurethane, allylurethane, and
R² independently represents a straight or branched C₆-C₄₀-alkyl group, a phenyl group, a polyphenyl group, preferably a straight or branched C₁₀-C₃₀-alkyl group, more preferentially a straight or branched C₁₂-C₂₂-alkyl group, or a group comprising 2 to 5 phenyl groups or a tristyrylphenyl group or a pentastyrylcumylphenyl group.

More particularly preferably according to the invention, the compound (C) is a compound of formula (I) wherein:
m independently represents an integer or decimal less than 150, n is equal to 0,
EO independently represents a CH₂CH₂O group,
R¹ independently represents a group chosen among an acrylate group, a methacrylate group, an unsaturated urethane group, in particular acrylurethane, methacrylurethane, α-α′-dimethyl-isopropenyl-benzylurethane, allylurethane, and
R² independently represents a straight or branched C₁₀-C₂₂-alkyl group, or a group comprising 2 to 5 phenyl groups or a tristyrylphenyl group or a pentastyrylcumylphenyl group.

In addition to the monomers (A), (B), optionally (C), the copolymer (b) may be prepared by a polymerisation reaction also using:
D—at least one compound (D) chosen among 2-acrylamido-2-methylpropane sulphonic acid (AMPS), ethoxy methacrylate sulphonic acid, sodium methallyl sulphonate, styrene sulphonate hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, their salts and combinations thereof; or
E—at least one compound (E) chosen among hydroxyethyl acrylate, hydroxypropyl acrylate, hydroxyethylhexyl acrylate, hydroxyethyl methacrylate, hydroxypropyl methacrylate, hydroxyethylhexyl methacrylate and combinations thereof; or
F—at least one monomer (F) chosen among a cross-linking monomer and a monomer comprising at least two olefinic unsaturations.

Preferably according to the invention, the compound (D) is chosen among 2-acrylamido-2-methylpropane sulphonic acid, its salts and combinations thereof.

More particularly preferably according to the invention, the monomer (F) is chosen among:

a compound of formula (II):

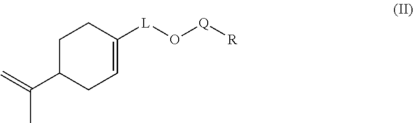

wherein:

L represents CH₂, CH₂ monoalkoxylate or CH₂ polyalkoxylate, preferably a CH₂ monoethoxylate group or a CH₂ polyethoxylate group;
Q represents a direct bond or C(O);
R represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, Q³OQ⁴OC(O)C(CH₃)═CH₂ or Q³OQ⁴OC(O)C(H)═CH₂;
Q³ represents a difunctional residue of an asymmetric diisocyanate compound, preferably chosen among tolyl-1,3-diisocyanate (TDI) and isophorone-diisocyanate (IPDI);
Q⁴ represents CH₂, CH₂—CH₂, CH₂ monoalkoxylate, CH₂—CH₂ monoalkoxylate, CH₂ polyalkoxylate or CH₂—CH₂ polyalkoxylate;
a compound of formula (III):

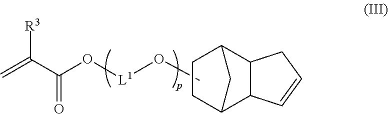

wherein:
R³ independently represents H or CH₃,
L¹ independently represents a straight or branched C₁-C₂₀-alkylene group preferably an ethylene group or a propylene group, and
p independently represents 0 or an integer ranging from 1 to 30, for example from 1 to 20, particularly from 1 to 15, in particular from 1 to 10.

The monomer (F) may also be chosen among di(meth)acrylates such as polyalkylene glycol di(meth)acrylate, in particular polypropylene glycol di(meth)acrylate, ethylene glycol di(meth)acrylate, polyethylene glycol di(meth)acrylate, triethylene glycol di(meth)acrylate, 1,3-butylene glycol di(meth)acrylate, 1,6-butyl ene glycol di(meth)acrylate, 1,6-hexanediol di(meth)acrylate, neopentyl glycol di(meth)acrylate, 1,9-nonanediol di(meth)acrylate, but also 2,2′-bis(4-(acryloxy-propyloxyphenyl))propane, 2,2′-bis(4-(acryloxydiethoxy-phenyl))propane and zinc acrylate; tri(meth)acrylate compounds such as trimethylolpropane tri(meth)acrylate and ethoxylated trimethylolpropane tri(meth)acrylate, trimethylolethane tri(meth)acrylate, pentaerythritol tri(meth)acrylate and tetramethylolmethane tri(meth)acrylate; tetra(meth)acrylate compounds such as di-trimethylolpropane tetra(meth)acrylate, tetramethylolmethane tetra(meth)acrylate and pentaerythritol tetra(meth)acrylate; hexa(meth)acrylate compounds such as dipentaerythritol hexa(meth)acrylate; penta(meth)acrylate compounds such as dipentaerythritol penta(meth)acrylate; allyl compounds such as allyl (meth)acrylate, diallyl phthalate, diallyl itaconate, diallyl fumarate, diallyl maleate; polyallyl carbohydrate ethers with from 2 to 8 groups per molecule, pentaerythritol polyallyl ethers such as pentaerythritol diallyl ether, pentaerythritol triallyl ether and pentaerythritol tetraallyl ether; trimethylolpropane polyallyl ethers such as trimethylolpropane diallyl ether and trimethylolpropane triallyl ether. Other polyunsaturated compounds include divinyl glycol, divinyl benzene, divinylcyclohexyl, and methylenebisacrylamide.

Monomer (F) can also be prepared by an esterification reaction of a polyol with an unsaturated anhydride such as acrylic anhydride, methacrylic anhydride, maleic anhydride, or itaconic anhydride. To obtain monomer (F), compounds chosen among polyhaloalkanols may also be used such as 1,3-dichloroisopropanol and 1,3-dibromoisopropanol; haloepoxyalkanes such as epichlorohydrin, epibromohydrin, 2-methyl epichlorohydrin and epiiodohydrin; polyglycidyl ethers such as 1,4-butanediol diglycidyl ether, glycerine-1,3-diglycidyl ether, ethylene glycol diglycidyl ether, propylene glycol diglycidyl ether, diethylene glycol diglycidyl ether, neopentyl glycol diglycidyl ether, polypropylene glycol diglycidyl ether, bisphenol A-epichlorohydrin epoxy resin and mixtures thereof.

Monomer (F) can also be a mixture of two separate monomers, such as EGDCPEA (ethylene glycol dicyclopentenyl ether acrylate) and TMPTA or EGDCPEA and TMPTA 3EO or even EGDCPEMA (ethylene glycol dicyclopentenyl ether methacrylate) and TMPTA or EGDCPEMA and TMPTA 3EO.

According to the invention, monomer (F) is preferably chosen among a compound of formula (II), a compound of formula (III), trimethylolpropane trimethacrylate, trimethylolpropane triacrylate, ethoxylated trimethylolpropane trimethacrylate, ethoxylated trimethylolpropane triacrylate, ethylene glycol dimethacrylate, ethylene glycol diacrylate, methylene bisacrylamide, diallyl phthalate, diallyl maleate, trimethylolpropane diallyl ether and mixtures thereof.

Particularly preferably according to the invention, the ASE copolymer (b1) is prepared by a polymerisation reaction using the monomers (A), (B) and (F).

Also particularly preferably according to the invention, the HASE copolymer (b2) is prepared by a polymerisation reaction using the monomers (A), (B), (C) and (F).

Also preferably, the polymerisation reaction uses:
from 20 to 70% by weight or from 30 to 60% by weight, preferably from 30 to 55% by weight or from 30 to 50% by weight, of monomer (A),
from 30 to 80% by weight or from 40 to 70% by weight, preferably from 45 to 70% by weight or from 50 to 70% by weight, of monomer (B),

relative to the total amount by weight of monomers (A) and (B).

Also advantageously according to the invention, the polymerisation reaction can use less than 30% by weight, preferably from 0.2 to 25% by weight, particularly from 0.5 to 20% by weight or from 0.5 to 15% by weight, of monomer (C) relative to the total amount by weight of monomers.

Also advantageously, the polymerisation reaction can use less than 20% by weight, preferably from 0.2 to 20% by weight, particularly from 0.5 to 10% by weight, of monomer (D) relative to the total amount by weight of monomers.

Also advantageously according to the invention, the polymerisation reaction can use less than 20% by weight, preferably from 0.2 to 20% by weight, particularly from 0.5 to 10% by weight, of monomer (E) relative to the total amount by weight of monomers.

Also advantageously, the polymerisation reaction can use less than 5% by weight, preferably from 0.01 to 4% by weight, particularly from 0.02 to 4% by weight or from 0.02 to 2% by weight, in particular from 0.02 to 1% by weight, of monomer (F) relative to the total amount by weight of monomers.

The copolymers (b1) and (b2) according to the invention are prepared by methods known as such. In particular, the copolymers (b1) and (b2) according to the invention may be prepared by a radical polymerisation reaction, for example a polymerisation reaction in emulsion, dispersion or solution. The polymerisation can be carried out in the presence of at least one initiator compound. As examples of initiator compounds, at least one compound may be used chosen among azoic initiator compounds (for example, azobisisobutyronitrile), a peroxide compound, preferably hydrogen peroxide, benzoyl peroxide, benzoyl hydroperoxide, and mixtures thereof. Alkaline metal persulphates can also be mentioned, particularly sodium persulphate and potassium persulphate, ammonium persulphate, partially water-soluble peroxides, particularly succinic peracid, t-butyl hydroperoxide, cumyl hydroperoxide, persulphates combined with a copper ion, a ferrous ion, a sulphite ion or a bisulphite ion and mixtures thereof.

Aside from the various monomers, the method for preparing the copolymers (b1) and (b2) generally uses at least one chain transfer agent, preferably chosen among the mercaptan compounds, in particular mercaptan compounds comprising at least four carbon atoms such as butyl mercaptan, n-octyl mercaptan, n-dodecyl mercaptan, tert-dodecyl mercaptan, iso-octyl 3-mercaptopropionate. Preferably, the reaction is a radical polymerisation reaction in an emulsion.

Preferably according to the invention, the copolymer (b) has a pH greater than 5, preferably a pH greater than 5.5, more preferentially a pH greater than 6 or a pH greater than 6.5. The copolymer (b) generally has a pH of less than 8.

Also preferably according to the invention, the copolymer (b) is totally or partially neutralised by means of a base. More preferably according to the invention, the copolymer (b) is totally or partially neutralised by means of an alkali-metal derivative or an alkaline-earth metal derivative. The preferred compounds are chosen among bases such as NaOH, KOH, NH₄OH, Ca(OH)₂, monoisopropylamine (AMP), triethylamine, diethylamine, monoethylamine.

Preferably according to the invention, the composition according to the invention also comprises at least one preservative compound (c). More preferably according to the invention, the preservative compound (c) is chosen among phenoxyethanol; 5-bromo-5-nitro-1,3-dioxane; behentrimonium chloride; benzalkonium chloride; benzoic acid; benzyl alcohol; cetrimonium bromide; cetrimonium chloride; chlorhexidine digluconate; chlorphenesin; climbazole; dehydroacetic acid; diazolidinyl urea; DMDM hydantoin (CAS number 6440-58-0); ethyl lauroyl arginate HCl; formaldehyde; formic acid; hexamidine diisethionate; imidazolidinyl urea; iodopropynyl butylcarbamate; isobutylparaben; isopropylparaben; methylchloroisothiazolinone; methylisothiazolinone; methylparaben; ethylparaben; propylparaben; butylparaben; myrtrimonium bromide; O-cymen-5-ol; piroctone olamine; polyaminopropyl biguanide; potassium benzoate; potassium sorbate; salicylic acid; silver chloride; sodium benzoate; sodium bisulphite; sodium dehydroacetate; sodium formate; sodium hydroxymethylglycinate; sodium metabisulphite; sodium methylparaben; sodium propylparaben; sodium salicylate; sodium sorbate; sodium sulphite; sorbic acid; stearalkonium chloride; triclosan; zinc pyrithione. Particularly preferably according to the invention, the preservative compound (c) is phenoxyethanol.

Within the composition according to the invention, the amounts of the various ingredients may vary. Preferably according to the invention, the composition according to the invention comprises, relative to the total weight of composition:
from 0.2 to 30% by dry weight of water-soluble compound (a);
from 0.3 to 15% by dry weight of copolymer (b);
from 0.001 to 5% by dry weight of preservative compound (c);
an additional quantity (qsp) of water.

Also preferably according to the invention, the composition according to the invention comprises, relative to the total weight of composition:
from 0.2 to 20% by dry weight of water-soluble compound (a);
from 0.3 to 10% by dry weight of copolymer (b);
from 0.001 to 3% by dry weight of preservative compound (c);
an additional quantity (qsp) of water.

The composition according to the invention can have different forms. Preferably, the composition according to the invention is liquid or gelled. More preferably, the composition according to the invention has a Brookfield viscosity, measured at 25° C. and at 20 rpm, ranging from 50 to 200,000 mPa·s.

The composition according to the invention is a cosmetic cleansing composition. Preferably, the composition according to the invention is a makeup removal composition. The composition according to the invention can be used alone or used directly due to its cleansing properties, in particular due to its makeup removing properties.

The composition according to the invention can also be used indirectly, particularly by incorporating it into a formulation, in particular into a cosmetic formulation.

Thus, the invention provides a cosmetic formulation comprising at least one composition according to the invention and at least one substance (d) chosen among a cosmetic admixture, a cosmetic additive and combinations thereof. Preferably according to the invention, the substance (d) is present in the hydrophilic phase. Also preferably according to the invention, the substance (d) is chosen among a fragrance, optionally combined with a fragrance dissolution agent, a preservative agent, a bactericidal agent, a colouring agent, a softening treatment agent, a buffer agent, a sunscreen, a UV filter, an electrolyte compound such as sodium chloride, a pH-adjusting compound, for example citric acid or sodium hydroxide, and combinations thereof.

The formulation according to the invention may also comprise a hydrophobic phase, in particular an oily phase.

The formulation according to the invention can be in different forms. In particular, the formulation according to the invention can be in biphasic form. Preferably, the biphasic formulation according to the invention comprises an aqueous phase comprising a composition according to the invention and a hydrophobic phase, in particular an oily phase.

The composition according to the invention or the formulation according to the invention can be used in various ways. In particular, this composition or formulation can be used as part of a cleansing method, preferably as part of a makeup removal method. Thus, the invention provides a method for cleansing a substrate comprising the application of at least one composition according to the invention or the application of at least one formulation according to the invention, to the substrate.

Preferably according to the invention, the cleansing method according to the invention, particularly the makeup removal method according to the invention, is used on a substrate chosen among a keratin substrate. More preferably according to the invention, the substrate is chosen among skin, hair and body hair.

Particularly preferably according to the invention, the cleansing method according to the invention, in particular the makeup removal method according to the invention, is used without rinsing, in particular without rinsing with water.

The particular, advantageous or preferred characteristics of the cosmetic composition according to the invention define formulations or methods according to the invention which are also particular, advantageous or preferred.

The following examples illustrate the various aspects of the invention.

### EXAMPLE 1: PREPARATION OF COPOLYMERS (B1) AND (B2) AND OF COMPOSITIONS ACCORDING to the Invention

Preparation of Copolymers (b1) and (b2):

In a 1 L reactor that is stirred and heated using an oil bath, a mixture (M1) is prepared by introducing deionised water, an anionic surfactant compound which is sodium lauryl sulphate (SLS) or sodium laureth sulphate (SLES—Texapon NSO UP at 28% by weight in water) and, optionally, a non-ionic surfactant compound (Rhodasurf ID 030 by Solvay or Polyglycol B01/40 by Clariant) and, optionally, a monomer (D) which is 2-acrylamido-2-methylpropane sulphonic acid (AMPS 2405 by Lubrizol) at 50% by weight in water.

In a beaker, a mixture (M2) is also prepared, comprising, in deionised water:
monomer (A): methacrylic acid (MAA),
monomer (B): ethyl acrylate (EA),
optionally, another monomer (B): methyl methacrylate (MMA),
optionally, a monomer (F): a cross-linking monomer (Fancryl 512-AS from Hitachi Chemical),
optionally a surfactant compound: SLS,
optionally a monomer (C) of formula (I): branched associative monomer C₁₆-EO₂₅-alkyl methacrylate or branched associative monomer C₁₂-EO₂₅-alkyl methacrylate or straight associative monomer C₂₂-EO₂₅-alkyl methacrylate or branched associative monomer C₂₀-EO₃₆-alkyl methacrylate,
optionally n-dodecylmercaptan.

The mixture (M2) is stirred to form a monomer mixture.

An initiator solution (S1) is prepared comprising ammonium persulphate and deionised water. Optionally, an initiator solution (S2) is prepared comprising sodium metabisulphite and deionised water. All reagents and amounts used are listed in Table 1.

For copolymers A, C and E, in the reactor heated to the polymerisation temperature±1° C., the initiator solution (S1) and the mixture (M2) are simultaneously injected over 2 hours (2.5 hours for copolymer C). For copolymers B, D and F, the initiator solutions (S1) and (S2) then the mixture (M2) are injected over 2 hours into the reactor heated to the polymerisation temperature±1° C.

Cook for 30 minutes at the same temperature. The mixture is then cooled to room temperature.

The polymers according to the invention were prepared under these conditions while varying the monomer compositions of the monomer mixture (M2). The copolymer compositions obtained are shown in Table 1.

| Copolymer/quantity (g): | | A | B | C | D | E | F |
|---|---|---|---|---|---|---|---|
| M1 | Deionised water | 575.00 | 492.46 | 477.00 | 384.50 | 257.00 | 479.70 |
| | SLS | 0.00 | 5.70 | 0.30 | 5.20 | 3.70 | 4.40 |
| | Texapon NSO UP | 10.50 | 0.00 | 0.00 | 0.00 | 0.00 | 0.00 |
| | Rhodasurf ID 030 | 0.00 | 0.00 | 0.00 | 4.40 | 0.00 | 4.20 |
| | AMPS (AMPS 2405) | 0.00 | 0.00 | 0.00 | 6.60 | 0.00 | 9.50 |
| | Polyglycol B01/40 | 0.00 | 5.30 | 0.00 | 0.00 | 0.00 | 0.00 |
| M2 | Deionised water | 25.30 | 184.46 | 93.10 | 125.36 | 340.00 | 130.54 |
| | SLS | 0.00 | 2.00 | 3.10 | 1.80 | 3.70 | 1.50 |
| | MAA | 88.16 | 100.00 | 105.90 | 83.64 | 111.90 | 120.60 |
| | EA | 176.30 | 160.90 | 200.20 | 123.30 | 83.00 | 164.10 |
| | MMA | 0.00 | 0.00 | 0.00 | 0.00 | 83.00 | 0.00 |
| | Fancryl 512-AS cross-linking | 0.90 | 0.00 | 0.60 | 0.00 | 0.00 | 0.00 |
| | monomer | | | | | | |
| | Branched C₁₂(EO)₂₅-alkyl | 0.00 | 0.00 | 0.00 | 28.20 | 0.00 | 0.00 |
| | methacrylate | | | | | | |
| | Branched C₁₆(EO)₂₅-alkyl | 22.95 | 0.00 | 0.00 | 0.00 | 29.00 | 0.00 |
| | methacrylate | | | | | | |
| | Straight C₂₂(EO)25-alkyl | 0.00 | 29.45 | 0.00 | 0.00 | 0.00 | 0.00 |
| | methacrylate | | | | | | |
| | Branched C₂₀(EO)₃₆-alkyl | 0.00 | 0.00 | 0.00 | 0.00 | 0.00 | 5.80 |
| | methacrylate | | | | | | |
| | N-dodecyl mercaptan | 0.09 | 0.00 | 0.00 | 0.26 | 0.90 | 0.00 |
| S1 | Deionised water | 62.8 | 1.25 | 46.3 | 5.80 | 39 | 14.00 |
| | Ammonium persulphate | 0.4 | 0.90 | 0.3 | 0.70 | 0.4 | 0.90 |
| S2 | Deionised water | 0.00 | 1.25 | 0.00 | 3.60 | 0.00 | 4.00 |
| | Sodium metabisulphite | 0.00 | 0.09 | 0.00 | 0.07 | 0.00 | 0.09 |

| | | | | | | |
|---|---|---|---|---|---|---|
| Polymerisation temperature | 85° C. | 76° C. | 87° C. | 76° C. | 85° C. | 75° C. |

Preparation and Evaluation of Compositions According to the Invention and Comparative Compositions

To prepare the compositions according to the invention, the ingredients are mixed under stirring using a Rayneri stirrer, then the pH of the formulation is adjusted by adding a 20% NaOH solution. The ingredients and amounts used are shown in Table 2.

The makeup-removing effects are evaluated for the compositions according to the invention and for the comparative compositions (C1) (Gamier All-in-One micellar solution) and (C2) (Nivea MicellAIR micellar gel).

To evaluate the cleansing efficacy of the compositions, a piece of Transpore 3M adhesive tape is glued to a smooth surface.

Then, 3 cosmetic products are applied on these pieces of adhesive tape: lipstick (Velvet Mat Red & Long lasting hold up to 24 h, 08 Rubi's cute by Bourgeois), eye pencil (Super liner Le Khol 101 Midnight Black by L'Oreal) and foundation (Super Stay 24 h 48 hours Sun beige by Maybelline New York). The tape is left to dry.

An amount of approximately 1 g of cleansing composition is applied to a cotton pad (Duo Pro by Inell, Leclerc reference brand).

The makeup product is manually wiped from the surface of the adhesive tape.

The cleansing efficacy of each composition is visually assessed according to the residual colour on each piece of adhesive tape after wiping and an efficacy score is assigned using a scale ranging from 1 to 4: 1 corresponds to very low cleansing efficacy, 2 corresponds to low cleansing efficacy, 3 corresponds to good cleansing efficacy and 4 corresponds to excellent cleansing efficacy.

Comparative composition (C1) obtains a score of 1 for lipstick removal, a score of 1 for eye pencil removal, and a score of 1 for foundation removal. Comparative composition (C2) obtains a score of 2 for lipstick removal, a score of 2 for eye pencil removal, and a score of 2 for foundation removal. The results obtained for the compositions according to the invention are shown in Tables 2 and 3.

| Composition according to the invention: | | 1 | 2 | 3 | 4 | 5 |
|---|---|---|---|---|---|---|
| Copolymer | Polymer A (ES = 28%) | 6 | 6 | 6 | 4 | 8 |
| Moistening | Glycerine (Sigma-Aldrich) | 1 | 1 | 1 | 1 | 1 |
| compound | Methylpropanediol | 0 | 1 | 0 | 0 | 0 |
| | (DUB Diol, Stéarinerie Dubois) | | | | | |
| | Pentylene glycol | 0 | 0 | 1 | 1 | 1 |
| | (Pentiol Green + Minnasolve) | | | | | |
| | Butylene glycol (Sigma-Aldrich) | 1 | 0 | 0 | 0 | 0 |
| Preservative | Phenoxyethanol (Sigma-Aldrich) | 1 | 1 | 1 | 1 | 1 |
| Water | | 91 | 91 | 91 | 93 | 89 |
| NaOH at 20% in water up to pH | | 6.7 | 6.8 | 6.9 | 6.9 | 6.9 |
| Brookfield viscosity at 20 rpm, 25° C. (mPa · s) | | 2,519 | 3,970 | 5,700 | 1,896 | 12,927 |
| Makeup-removing | lipstick | 4 | 4 | 4 | 4 | 4 |
| power | eye pencil | 4 | 4 | 4 | 4 | 4 |
| | foundation | 4 | 4 | 4 | 4 | 4 |

At various dosages, the composition according to the invention comprising the copolymer A makes it possible to obtain excellent results for removing lipstick, eye pencil and foundation. Comparative cleansing compositions do not make it possible to obtain such results.

| Composition according to the invention: | | 6 | 7 | 8 | 9 | 10 |
|---|---|---|---|---|---|---|
| Copolymer | Polymer B (ES = 30%) | 6 | 0 | 0 | 0 | 0 |
| | Polymer C (ES = 30%) | 0 | 6 | 0 | 0 | 0 |
| | Polymer D (ES = 30%) | 0 | 0 | 6 | 0 | 0 |
| | Polymer E (ES = 30%) | 0 | 0 | 0 | 6 | 0 |
| | Polymer F (ES = 30%) | 0 | 0 | 0 | 0 | 6 |
| Moistening | Glycerine (Sigma-Aldrich) | 1 | 1 | 1 | 1 | 1 |
| compound | Pentylene glycol (Pentiol Green +, | 1 | 1 | 1 | 1 | 1 |
| Preservative | Phenoxyethanol (Sigma-Aldrich) | 1 | 1 | 1 | 1 | 1 |
| Water | | 91 | 91 | 91 | 91 | 91 |
| NaOH at 20% in water up to pH | | 6.9 | 6.9 | 6.9 | 6.9 | 6.9 |
| Brookfield viscosity at 20 rpm, 25° C. (mPa · s) | | 100,000 | 4,200 | 630 | 113 | 28,524 |
| Makeup- | lipstick | 4 | 4 | 4 | 4 | 4 |
| removing | eye pencil | 3 | 3 | 3 | 4 | 4 |
| power | foundation | 4 | 4 | 4 | 4 | 4 |

Unlike the comparative cleansing compositions, the compositions according to the invention comprising different copolymers make it possible to remove lipstick, eye pencil and foundation with very good results or excellent results.

1. A water-based cosmetic cleansing composition that does not comprise any surfactant compound or any lipophilic compound, comprising:
(a) at least one water-soluble compound selected from the group consisting of a water-soluble moistening compound and a water-soluble solvent;
(b) at least one copolymer selected from the group consisting of:
(b1) an ASE copolymer, and
(b2) an HASE copolymer.

2. The composition according to claim 1, consisting of water;
(a) the at least one water-soluble compound;
(b) the at least one copolymer; and optionally
(c) at least one preservative compound.

3. The composition according to claim 1, wherein the at least one water-soluble compound (a) is different from ethanol or is selected from the group consisting of a synthetically-derived water-soluble compound, and a naturally-derived water-soluble compound.

4. The cleansing composition according to claim 1, wherein the at least one compound (b) is selected from the group consisting of:
an ASE copolymer (b1) prepared by at least one polymerisation reaction of at least one anionic monomer (A) and at least one ester (B) of an acid, wherein
the at least one anionic monomer (A) comprises at least one polymerisable olefinic unsaturation; and
the at least one ester (B) of an acid is selected from the group consisting of acrylic acid, methacrylic acid, maleic acid, maleic anhydride, itaconic acid and crotonic acid;
an HASE copolymer (b2) prepared by polymerisation reaction of at least one anionic monomer (A); at least one ester (B) and at least one compound (C) of formula (I):
R¹-(EO)ₘ—(PO)ₙ—R² (I)
wherein:
m and n independently represent 0 or an integer or decimal less than 150, m or n being different from 0,
EO independently represents a CH₂CH₂O group,
PO independently represents a group selected from the group consisting of CH(CH₃)CH₂O and CH₂CH(CH₃)O and combinations thereof,
R¹ independently represents a group comprising at least one polymerisable olefinic unsaturation, and
R² independently represents a straight or branched C₆-C₄₀-alkyl group, a phenyl group, a polyphenyl group.

5. The composition according to claim 1, wherein the at least one copolymer (b) has a pH greater than 5.

6. The composition according to claim 1, wherein the at least one copolymer (b) is totally or partially neutralised by means of a base.

7. The composition according to claim 1, wherein the at least one copolymer (b) is prepared by a polymerisation reaction which uses:
at least one compound (D) selected from the group consisting of 2-acrylamido-2-methylpropane sulphonic acid, ethoxymethacrylate sulphonic acid, sodium methallyl sulphonate, styrene sulphonate hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, and their salts; or
at least one compound (E) selected from the group consisting of hydroxyethyl acrylate, hydroxypropyl acrylate, hydroxyethylhexyl acrylate, hydroxyethyl methacrylate, hydroxypropyl methacrylate, and hydroxyethylhexyl methacrylate; or
at least one monomer (F) selected from the group consisting of a cross-linking monomer and a monomer comprising at least two olefinic unsaturations.

8. The composition according to claim 1, further comprising at least one preservative compound (c).

9. The composition according to claim 1, comprising, relative to the total weight of composition:
from 0.2 to 30% by dry weight or from 0.2 to 20% by dry weight of the at least one water-soluble compound (a);
from 0.3 to 15% by dry weight or from 0.3 to 10% by dry weight of the at least one copolymer (b);
from 0.001 to 5% by dry weight or from 0.001 to 3% by dry weight of a preservative compound (c);
an additional quantity (qsp) of water.

10. The composition according to claim 1, which is liquid or gelled.

11. The composition according to claim 1, which is a makeup removal composition.

12. A cosmetic formulation comprising the composition according to claim 1 and at least one substance (d) selected from the group consisting of a cosmetic admixture and a cosmetic additive.

13. The formulation according to claim 12 further comprising a hydrophobic phase.

14. The formulation according to claim 13 which is in biphasic form and comprising:
an aqueous phase comprising the composition, and
a hydrophobic phase.

15. A method for cleansing a substrate, the method comprising: applying the composition according to claim 1 on the substrate, optionally without rinsing with water.

16. The cleansing method according to claim 15, wherein the substrate is keratin.

17. The composition according to claim 1, wherein the at least one copolymer (b) has a pH less than 8.
